(19) United States
(12) Patent Application Publication
(10) Pub. No.: US 2023/0245308 A1
(43) Pub. Date: Aug. 3, 2023

(54) MEDICAL IMAGE PROCESSING APPARATUS, MEDICAL IMAGE PROCESSING METHOD AND NON-TRANSITORY COMPUTER-READABLE STORAGE MEDIUM

(71) Applicant: CANON KABUSHIKI KAISHA, Tokyo (JP)

(72) Inventors: Takeshi NODA, Kanagawa (JP); Sota TORII, Tokyo (JP); Haruki IWAI, Tokyo (JP); Yoshimasa KOBAYASHI, Tokyo (JP); Hisanori KATO, Tokyo (JP)

(21) Appl. No.: 18/192,832

(22) Filed: Mar. 30, 2023

Related U.S. Application Data
(63) Continuation of application No. PCT/JP2021/037378, filed on Oct. 8, 2021.

(30) Foreign Application Priority Data
Oct. 22, 2020 (JP) 2020-177426

(51) Int. Cl. G06T 7/00; A61B 6/00

(52) U.S. Cl. CPC G06T 7/0012; A61B 6/52; G06T 2207/10116

(57) ABSTRACT

A medical image processing apparatus comprising: a setting unit configured to set a plurality of candidate regions on a medical image as candidates of a region of interest to be set on the medical image; an analysis unit configured to execute quantitative analysis concerning a composition of an object for each of the plurality of candidate regions; and an output unit configured to output a plurality of analysis results and the plurality of candidate regions in association with each other.

### CROSS-REFERENCE TO RELATED APPLICATIONS

This application is a Continuation of International Patent Application No. PCT/JP2021/037378, filed Oct. 8, 2021, which claims the benefit of Japanese Patent Application No. 2020-177426, filed Oct. 22, 2020, both of which are hereby incorporated by reference herein in their entirety.

### BACKGROUND OF THE INVENTION

### Field of the Invention

Embodiments disclosed in this specification relate to a medical image processing apparatus, a medical image processing method and a non-transitory computer-readable storage medium.

### Background Art

Techniques of performing quantitative analysis for a region of interest set on a medical image have become popular. As the techniques of performing quantitative analysis, for example, BMD (Bone Mineral Density), BD (Breast Density), and the like are known. A result of quantitative analysis may be affected by the set region of interest.

One of problems to be solved by the embodiments disclosed in this specification is to set an appropriate region of interest. However, the problem to be solved by the embodiments disclosed in this specification is not limited to the above-described problem. It is also possible to define a problem corresponding to each effect by each configuration shown in embodiments to be described later as another problem to be solved by the embodiments disclosed in this specification.

### CITATION LIST

### Patent Literature

PTL 1: Japanese Patent Laid-Open No. 2008-104798

### SUMMARY OF THE INVENTION

A medical image processing apparatus according to an embodiment includes a medical image processing apparatus comprising: a setting unit configured to set a plurality of candidate regions on a medical image as candidates of a region of interest to be set on the medical image; an analysis unit configured to execute quantitative analysis concerning a composition of an object for each of the plurality of candidate regions; and an output unit configured to output a plurality of analysis results and the plurality of candidate regions in association with each other.

Further features of the present invention will become apparent from the following description of exemplary embodiments with reference to the attached drawings.

### BRIEF DESCRIPTION OF THE DRAWINGS

FIG. 1 is a block diagram showing an example of the configuration of a medical information processing system according to the first embodiment.

FIG. 2 is a view for explaining bone mineral density measurement according to the first embodiment.

FIG. 3A is a view for explaining bone mineral density measurement according to the first embodiment.

FIG. 3B is a view for explaining bone mineral density measurement according to the first embodiment.

FIG. 3C is a view for explaining bone mineral density measurement according to the first embodiment.

FIG. 4 is a view for explaining bone mineral density measurement according to the first embodiment.

FIG. 5 is a view for explaining candidate regions according to the first embodiment.

FIG. 6 is a view showing a display example according to the first embodiment.

FIG. 7 is a view showing a display example according to the first embodiment.

FIG. 8 is a view showing a display example according to the first embodiment.

FIG. 9 is a view showing a display example according to the first embodiment.

FIG. 10 is a view showing a display example according to the first embodiment.

FIG. 11 is a flowchart for explaining a series of procedures of processing of a medical image processing apparatus according to the first embodiment.

FIG. 12 is a view for explaining breast density measurement according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

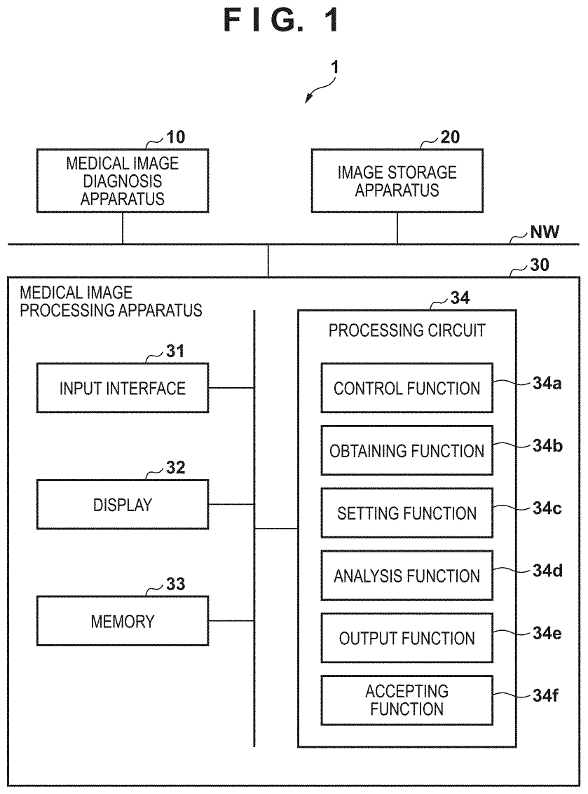

A medical information processing system 1 including a medical image diagnosis apparatus 10, an image storage apparatus 20, and a medical image processing apparatus 30 will be described as an example. For example, the medical image diagnosis apparatus 10, the image storage apparatus 20, and the medical image processing apparatus 30 are connected to each other via a network NW, as shown in FIG. 1.

The medical image diagnosis apparatus 10 is an apparatus that performs imaging of an object P and collects a medical image. If the medical image diagnosis apparatus 10 is an X-ray diagnosis apparatus, the medical image diagnosis apparatus 10 irradiates the object P with X-rays and detects the X-rays transmitted through the object P, thereby collecting X-ray images. Also, the medical image diagnosis apparatus 10 transmits the collected X-ray images to the image storage apparatus 20 or the medical image processing apparatus 30 via the network NW.

The image storage apparatus 20 stores various kinds of medical images. For example, the image storage apparatus 20 accepts an X-ray image collected by the medical image diagnosis apparatus 10 and stores it in a memory provided in the apparatus or outside the apparatus. For example, the image storage apparatus 20 is a server of a Picture Archiving and Communication System (PACS).

The medical image processing apparatus 30 executes various kinds of processing to be described later using the medical image collected by the medical image diagnosis apparatus 10. For example, the medical image processing apparatus 30 includes an input interface 31, a display 32, a memory 33, and a processing circuit 34, as shown in FIG. 1.

The input interface 31 accepts various kinds of input operations from a user, converts each accepted input operation into an electrical signal, and outputs it to the processing circuit 34. For example, the input interface 31 is implemented by a mouse, a keyboard, a trackball, a switch, a button, a joystick, a touch pad through which the user performs an input operation by touching the operation surface, a touch screen formed by integrating a display screen and a touch pad, a noncontact input circuit using an optical sensor, a voice input circuit, or the like. Note that the input interface 31 may be formed by a tablet terminal capable of wirelessly communicating with the main body of the medical image processing apparatus 30. Also, the input interface 31 may be a circuit that accepts the input operation from the user by motion capture. As an example, the input interface 31 processes a signal obtained via a tracker or an image collected for the user, thereby accepting the body motion or line of sight of the user as an input operation. The input interface 31 is not limited to a device including a physical operation component, such as a mouse or a keyboard. For example, an electrical signal processing circuit that accepts an electrical signal corresponding to an input operation from an external input device provided separately from the medical image processing apparatus 30 and outputs the electrical signal to the processing circuit 34 is also included in the example of the input interface 31.

The display 32 displays various kinds of information. For example, under the control of the processing circuit 34, the display 32 displays a medical image collected by the medical image diagnosis apparatus 10 or a result of analysis executed using the medical image. Also, for example, the display 32 displays a Graphical User Interface (GUI) configured to accept various kinds of instructions or settings from the user via the input interface 31. For example, the display 32 is a liquid crystal display or a Cathode Ray Tube (CRT) display. The display 32 may be of a desktop type or may be formed by a tablet terminal capable of wirelessly communicating with the main body of the medical image processing apparatus 30.

Note that in FIG. 1, a description will be made assuming that the medical image processing apparatus 30 includes the display 32. However, the medical image processing apparatus 30 may include a projector in place of or in addition to the display 32. Under the control of the processing circuit 34, the projector can perform projection to a screen, a wall, a floor, or the body surface of the object P. As an example, the projector can perform projection to an arbitrary plane, an object, or a space by projection mapping.

The memory 33 is implemented by, for example, a semiconductor memory element such as a Random Access Memory (RAM) or a flash memory, a hard disk, an optical disk, or the like. For example, the memory 33 stores a program for a circuit included in the medical image processing apparatus 30 to implement its function. Also, the memory 33 stores medical images collected by the medical image diagnosis apparatus 10. Note that the memory 33 may be implemented by a server group (cloud) connected to the medical image processing apparatus 30 via the network NW.

The processing circuit 34 executes a control function 34a, an obtaining function 34b, a setting function 34c, an analysis function 34d, an output function 34e, and an accepting function 34f, thereby controlling the operation of the entire medical image processing apparatus 30. The obtaining function 34b is an example of an obtaining unit. The setting function 34c is an example of a setting unit. The analysis function 34d is an example of an analysis unit. The output function 34e is an example of an output unit. The accepting function 34f is an example of an accepting unit.

For example, the processing circuit 34 reads out a program corresponding to the control function 34a from the memory 33 and executes it, thereby controlling various kinds of functions such as the obtaining function 34b, the setting function 34c, the analysis function 34d, the output function 34e, and the accepting function 34f based on various kinds of input operations accepted from the user via the input interface 31.

Also, for example, the processing circuit 34 reads out a program corresponding to the obtaining function 34b from the memory 33 and executes it, thereby obtaining a medical image. For example, the obtaining function 34b obtains, via the network NW, an X-ray image collected by the medical image diagnosis apparatus 10 and stored in the image storage apparatus 20. Alternatively, the obtaining function 34b may directly obtain an X-ray image from the medical image diagnosis apparatus 10 without interposing the image storage apparatus 20.

Additionally, for example, the processing circuit 34 reads out a program corresponding to the setting function 34c from the memory 33 and executes it, thereby setting a plurality of candidate regions on a medical image as the candidates of a region of interest to be set on the medical image. Also, for example, the processing circuit 34 reads out a program corresponding to the analysis function 34d from the memory 33 and executes it, thereby executing quantitative analysis concerning the composition of the object P for each of the plurality of candidate regions. In addition, for example, the processing circuit 34 reads out a program corresponding to the output function 34e from the memory 33 and executes it, thereby outputting a plurality of analysis results corresponding to the plurality of candidate regions. Also, for example, the processing circuit 34 reads out a program corresponding to the accepting function 34f from the memory 33 and executes it, thereby accepting, from the user who has referred to the analysis results, an operation of selecting one of the plurality of candidate regions as a region of interest. Note that processes by the setting function 34c, the analysis function 34d, the output function 34e, and the accepting function 34f will be described later.

In the medical image processing apparatus 30 shown in FIG. 1, each processing function is stored in the memory 33 in a form of a program executable by a computer. The processing circuit 34 is a processor that reads out the program from the memory 33 and executes it, thereby implementing the function corresponding to each program. In other words, the processing circuit 34 that has read out a program has a function corresponding to the readout program.

Note that in FIG. 1, the description has been made assuming that the single processing circuit 34 implements the control function 34a, the obtaining function 34b, the setting function 34c, the analysis function 34d, the output function 34e, and the accepting function 34f. However, the processing circuit 34 may be formed by combining a plurality of independent processors, and each processor may execute a program to implement the function. Also, the processing functions of the processing circuit 34 may be implemented by appropriately being distributed to or integrated in a single or a plurality of processing circuits.

In addition, the processing circuit 34 may implement the functions using the processor of an external apparatus connected via the network NW. For example, the processing circuit 34 reads out a program corresponding to each function from the memory 33 and executes it and also uses, as a calculation resource, a server group (cloud) connected to the medical image processing apparatus 30 via the network NW, thereby implementing each function shown in FIG. 1.

An example of the configuration of the medical information processing system 1 including the medical image processing apparatus 30 has been described above. Under this configuration, the medical image processing apparatus 30 sets a region of interest on an X-ray image collected by the medical image diagnosis apparatus 10 and executes quantitative analysis according to the region of interest.

For example, the medical image processing apparatus 30 performs bone mineral density measurement as the quantitative analysis, and calculates a Bone Mineral Density (BMD). In this case, in the medical image diagnosis apparatus 10, using a plurality of X-ray energies, an X-ray image corresponding to each X-ray energy is collected. For example, the medical image diagnosis apparatus 10 executes dual energy collection and collects a first X-ray image corresponding to a first X-ray energy and a second X-ray image corresponding to a second X-ray energy. As an example, the medical image diagnosis apparatus 10 can execute the dual energy collection by controlling a tube voltage supplied to an X-ray tube. Also, the medical image diagnosis apparatus 10 transmits the first X-ray image and the second X-ray image to the image storage apparatus 20 or the medical image processing apparatus 30 via the network NW.

Next, the obtaining function 34b obtains the first X-ray image and the second X-ray image from the image storage apparatus 20 or the medical image diagnosis apparatus 10. Next, the obtaining function 34b generates a bone density image based on the first X-ray image and the second X-ray image.

For example, the obtaining function 34b performs substance discrimination processing, thereby separating a bone component existing in a body part to be imaged from other components. As an example, the obtaining function 34b defines calcium (Ca) and water as reference substances, and estimates the existence ratio of calcium to water and the existence ratio of water to calcium on a pixel basis. This allows the obtaining function 34b to generate, as a bone density image, a substance discrimination image corresponding to the calcium component. Note that the description has been made assuming that the obtaining function 34b generates a bone density image. However, the obtaining function 34b may obtain, via the network NW, a bone density image generated by the medical image diagnosis apparatus 10 or another apparatus.

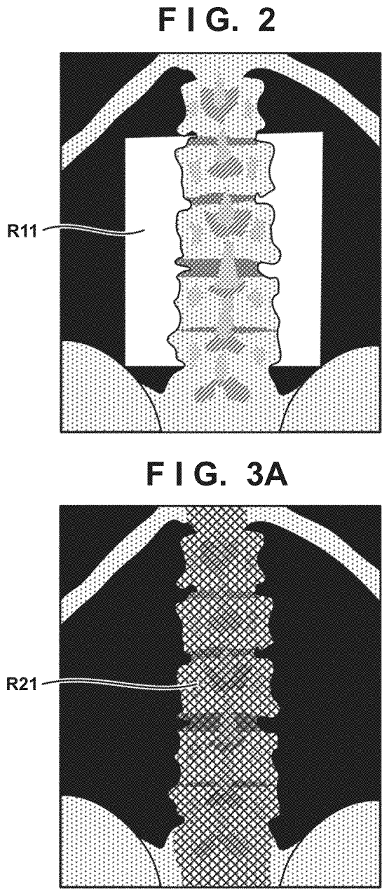

Next, the setting function 34c sets a region of interest on the bone density image. For example, the setting function 34c sets a region R11 of interest, as shown in FIG. 2. For example, the region R11 of interest is a soft tissue. The region R11 of interest may automatically be set by the setting function 34c or may be set based on a user operation. Note that FIG. 2 is a view for explaining bone mineral density measurement according to the first embodiment. A case where bone mineral density measurement for vertebrae is performed will be described with reference to FIG. 2.

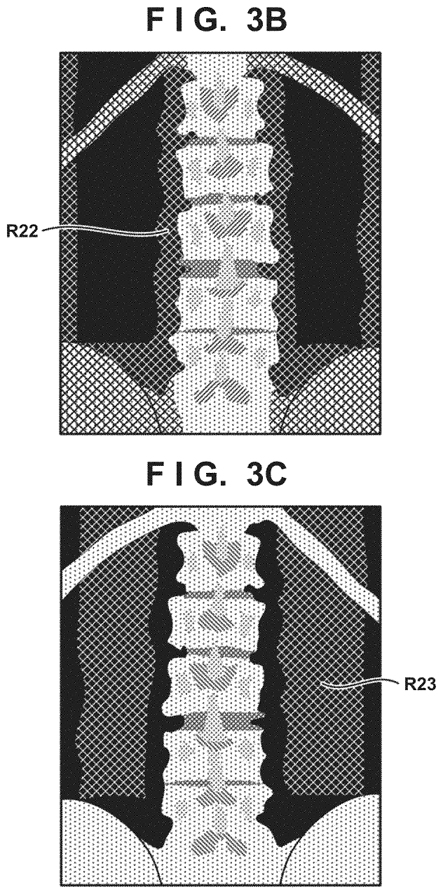

There is a risk that the region R11 of interest shown in FIG. 2 includes horizontal projections of vertebrae and bones (ribs, ilia, and the like) other than the vertebrae. In this case, the BMD may be calculated relatively low. The setting function 34c further sets neutral regions as regions of interest, as shown in FIGS. 3A, 3B, and 3C. FIGS. 3A, 3B, and 3C are views for explaining bone mineral density measurement according to the first embodiment.

For example, the setting function 34c sets a region R21 of interest corresponding to vertebrae, as shown in FIG. 3A. In addition, the setting function 34c sets a region R22 of interest corresponding to the horizontal projections of the vertebrae and bones other than the vertebrae, as shown in FIG. 3B. That is, the region R22 of interest is a neutral region corresponding to neither the vertebrae nor soft tissues. Also, the setting function 34c sets a region R23 of interest corresponding to soft tissues, as shown in FIG. 3C.

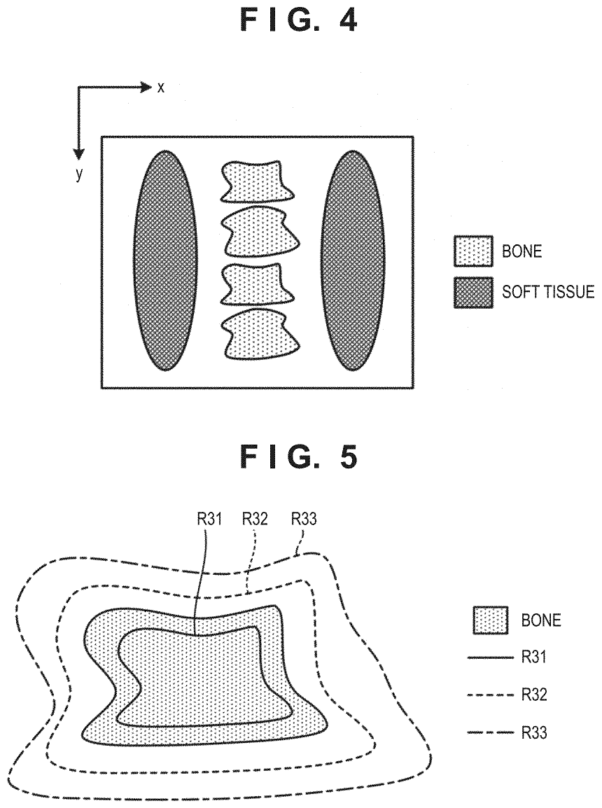

The analysis function 34d executes bone mineral density measurement based on the set regions of interest. A case where a region of interest corresponding to bones and a region of interest corresponding to soft tissues are set, as shown in FIG. 4, will be described below. FIG. 4 is a view for explaining bone mineral density measurement according to the first embodiment. In FIG. 4, the description will be made defining the abscissa of a bone density image as an x-axis and the ordinate as a y-axis.

When calculating a BMD, a region of interest that is set by extracting a bone region on a bone density image is used. Hence, the value of the BMD is affected by the region of interest. If the region of interest is not appropriately set, the BMD may have an inappropriate value. However, the boundary between tissues, for example, between a bone and a soft tissue is sometimes ambiguous, and it is not easy to set the region of interest. These exist several methods for setting a region of interest, and a method using Artificial Intelligence (AI) has also been proposed. However, none of the methods guarantees always setting an appropriate region of interest. The user can finely adjust a set region of interest in accordance with taste or a patient. However, it is not easy to grasp the influence of the result of fine adjustment on quantitative analysis.

The medical image processing apparatus 30 enables setting of an appropriate region of interest by processing to be described below in detail. More specifically, first, the obtaining function 34b obtains a medical image. For example, the obtaining function 34b generates a bone density image based on the first X-ray image and the second X-ray image obtained by dual energy collection. Alternatively, the obtaining function 34b obtains, via the network NW, a bone density image generated by another apparatus.

Next, the setting function 34c sets a plurality of candidate regions on the medical image as the candidates of a region of interest to be set on the medical image. This will be described below with reference to FIG. 5. FIG. 5 is a view for explaining candidate regions according to the first embodiment. FIG. 5 shows only a vertebra included in a bone density image for the descriptive convenience. For example, the setting function 34c sets a candidate region R31, a candidate region R32, and a candidate region R33 as the candidates of a region of interest corresponding to the vertebra, as shown in FIG. 5.

The setting function 34c can set a candidate region by various methods. For example, the setting function 34c can set a candidate region using a learned model that is activated to accept input of a medical image and estimate a region of interest. The learned model may be generated by the setting function 34c, or may be generated by another apparatus other than the medical image processing apparatus 30. The learned model is stored in, for example, the memory 33 and appropriately read out and used by the setting function 34c.

For example, the learned model is formed by a convolutional neural network. The convolutional neural network is a network that propagates information from an input layer side to an output layer side while maintaining the relationship between pixels. For example, a region of interest which is set on a medical image in the past and for which no abnormal value was generated in the result of bone mineral density measurement based on the region of interest is used as supervisory data. Setting the medical image to input side data and the supervisory data of the region of interest to output side data, learning is executed for a multilayer neural network, thereby generating a learned model with such a parameter that generates the supervisory data of the region of interest from the input side data. Note that the multilayer neural network is formed by, for example, an input layer, a plurality of intermediate layers (hidden layers), and an output layer.

The setting function 34c inputs the bone density image obtained by the obtaining function 34b to the learned model, and sets the estimation result of a region of interest obtained by the learned model to a candidate region.

Note that a plurality of learned models may be used, and a plurality of estimation results of regions of interest obtained by the learned models may be set to candidate regions. In this case, the learned models are generated based on supervisory data obtained by changing the strictness of region determination, the creator, or a group.

The setting function 34c may set a candidate region by threshold-based processing. For example, the setting function 34c may set, to a candidate region, a result of estimating a region corresponding to a bone by comparing the pixel value of each pixel in the bone density image obtained by the obtaining function 34b with a threshold.

Also, the setting function 34c may set a candidate region by performing graph cut processing for the bone density image.

The setting function 34c may set a candidate region by a user operation. For example, the output function 34e displays, on the display 32, the bone density image obtained by the obtaining function 34b, and the user performs an operation of drawing the outline of the candidate region while referring to the bone density image. In this case, the setting function 34c may set the region drawn by the user to a candidate region. The output function 34e displays a plurality of candidate regions on the display 32, and the user performs an operation of adjusting the displayed candidate regions. In this case, the setting function 34c sets the regions adjusted by the user to new candidate regions.

Also, the setting function 34c may set a candidate region by performing morphology processing. For example, the setting function 34c sets a candidate region by enlarging or reducing a region output from the learned model as the estimation result of a region of interest. As an example, the setting function 34c sets a region output from the learned model as the estimation result of a region of interest to the candidate region R32, sets a region obtained by reducing the candidate region R32 to the candidate region R31, and sets a region obtained by enlarging the candidate region R32 to the candidate region R33.

As an example, the setting function 34c accepts, from the user, an operation of selecting one of a plurality of parameters set in advance and performs morphology processing based on the selected parameter. For example, the setting function 34c accepts, from the user, an operation of selecting one of parameters “80%”, “90%”, “110%”, and “120%”. For example, if “110%” is selected, the setting function 34c enlarges the region output from the learned model as the estimation result of a region of interest to the size “110%” while maintaining the shape and the center position. Alternatively, the setting function 34c may perform morphology processing based on an arbitrary parameter accepted from the user. That is, the setting function 34c may perform discrete morphology processing or continuous morphology processing.

As another example, the output function 34e displays the bone density image on the display 32, and the user designates an arbitrary point on the bone density image. The setting function 34c enlarges or reduces the region output from the learned model as the estimation result of a region of interest such that the region passes through the point designated by the user, thereby setting a candidate region. For example, the output function 34e displays, on the display 32, the candidate region R31, the candidate region R32, and the candidate region R33 shown in FIG. 5. The user designates an arbitrary point in the region between the candidate region R31 and the candidate region R32 or in the region between the candidate region R32 and the candidate region R33. For example, if a point in the region between the candidate region R31 and the candidate region R32 is designated, the setting function 34c enlarges or reduces the candidate region R31 or the candidate region R32 such that the region passes through the point designated by the user, thereby setting a candidate region.

Note that a case where a candidate region is set by executing morphology processing for the region output from the learned model has been described. However, the embodiment is not limited to this. For example, the setting function 34c may set a candidate region by executing morphology processing for a region set by threshold-based processing, a region set by graph cut processing, or a region created by a user operation.

Furthermore, the setting function 34c may set a candidate region including a cortical bone and a candidate region that does not include the cortical bone. In general, since the bone density changes between a cortical bone and a cancellous bone inside the cortical bone, pixel values on the bone density image also change. Hence, the setting function 34c can extract a cortical bone region from the bone density image and set a candidate region including the cortical bone and a candidate region that does not include the cortical bone. The output function 34e may display a plurality of candidate regions such that it can be identified whether each candidate region includes the cortical bone.

The analysis function 34d executes bone mineral density measurement for each of the plurality of candidate regions set by the setting function 34c. For example, the analysis function 34d calculates a BMD for each of the candidate regions R31 to R33.

The output function 34e outputs the analysis result of the analysis function 34d. The output function 34e displays a plurality of analysis results and a plurality of candidate regions in association with each other. For example, the output function 34e displays, on the display 32, the plurality of candidate regions R31 to R33 and calculated BMDs in association with each other.

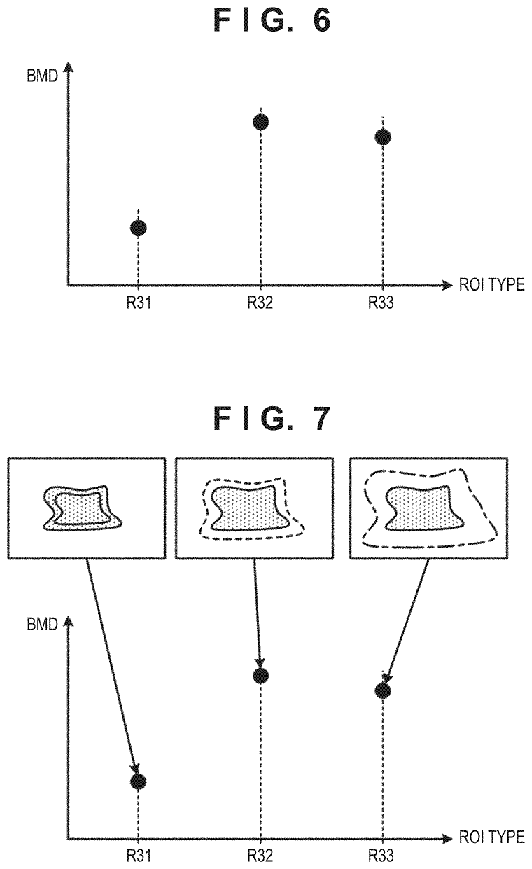

For example, the output function 34e displays, on the display 32, a graph in which the abscissa represents the types (ROI TYPE) of candidate regions such as the candidate region R31, the candidate region R32, and the candidate region R33, and the ordinate represents the value of each BMD, as shown in FIG. 6.

Based on the graph displayed on the display 32, the user determines whether to select a candidate region. The user roughly estimates the value of a BMD from various kinds of information including patient information such as the age and sex of the object P, a symptom that the object P complains of, a past inspection result for the object P, and an impression obtained by referring to the bone density image. The graph shown in FIG. 6 is a material to determine which candidate region is appropriate as a region of interest.

More specifically, the user refers to the display of the bone density image and the candidate regions shown in FIG. 5, and selects one of the candidate region R31, the candidate region R32, and the candidate region R33. Here, assume a case where although it can be determined that the candidate region R33 is not appropriate, it is difficult to determine which one of the candidate region R31 and the candidate region R32 is appropriate. In this case, the user can refer to the graph shown in FIG. 6 and thus select one of the candidate region R31 and the candidate region R32, which has a more appropriate BMD value, as a region of interest.

The accepting function 34f accepts an operation of selecting a region of interest. For example, the user performs the operation of selecting a region of interest by a click operation using a mouse, an operation of tapping on a touch panel, or voice input. For example, the accepting function 34f accepts, as the operation of selecting a region of interest, an operation of designating a position on the graph shown in FIG. 6 or a position on characters “R31”, “R32”, or “R33. Also, the accepting function 34f accepts, as the operation of selecting a region of interest, an operation of designating a position on the bone density image shown in FIG. 5. Furthermore, the accepting function 34f accepts, as the operation of selecting a region of interest, an operation of designating a position on an explanatory note shown in FIG. 5.

The output function 34e outputs an analysis result corresponding to the selected region of interest. For example, if the candidate region R32 is selected as a region of interest, the output function 34e displays, on the display 32, a report in which the BMD of the candidate region R32 is written. This allows the user to make various kinds of diagnoses such as grasping the effect of treatments to the object P and making a treatment plan.

Note that the above-described user may be either a doctor or a laboratory technician. For example, the accepting function 34f accepts selection of a region of interest from a doctor, and the output function 34e displays the analysis result corresponding to the selected region of interest to the doctor. Also, for example, the accepting function 34f accepts selection of a region of interest from a laboratory technician, and the output function 34e displays the analysis result corresponding to the selected region of interest to a doctor.

A case where the output function 34e controls display on the display 32 has been described. However, the embodiment is not limited to this. For example, the output function 34e may control a projector and project the graph shown in FIG. 6. Alternatively, for example, the output function 34e may transmit the graph shown in FIG. 6 to another apparatus, and the display of the other apparatus may perform display to the user. Alternatively, for example, the output function 34e may output the graph shown in FIG. 6 from a printer and provide it to the user. This also applies to various kinds of display to be described later.

The graph shown in FIG. 6 is merely an example, and various modification can be made. The output function 34e may display the BMD calculated based on the candidate region R31, the BMD calculated based on the candidate region R32, and the BMD calculated based on the candidate region R33 as a table or a text in association with the candidate region R31, the candidate region R32, and the candidate region R33, respectively.

Also, the output function 34e may further display an image representing a candidate region in association with an analysis result. As an example, the output function 34e displays an image representing the candidate region R31 on the graph in linkage with the plot of the BMD calculated based on the candidate region R31, as shown in FIG. 7. Similarly, the output function 34e displays an image representing the candidate region R32 on the graph in linkage with the plot of the BMD calculated based on the candidate region R32. Similarly, the output function 34e displays an image representing the candidate region R33 on the graph in linkage with the plot of the BMD calculated based on the candidate region R33.

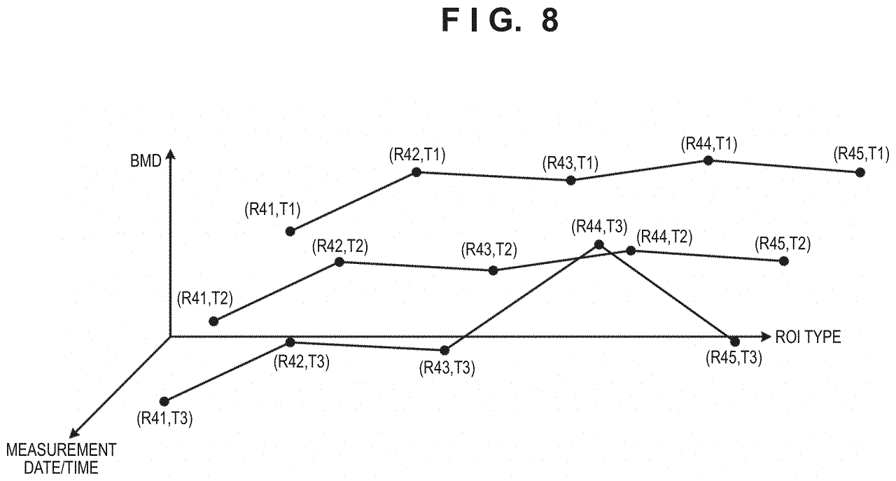

If bone mineral density measurement is performed for the object P a plurality of times, the output function 34e may display an analysis result corresponding to each of the plurality of candidate regions in association with the time base. This will be described below with reference to FIG. 8. FIG. 8 shows a case where bone mineral density measurement of the object P is performed at a measurement date/time T1, a measurement date/time T2, and a measurement date/time T3. For example, the setting function 34c sets candidate regions including a candidate region R41, a candidate region R42, a candidate region R43, a candidate region R44, and a candidate region R45 at each measurement date/time.

As an example, the setting function 34c inputs a bone density image collected from the object P at the measurement date/time T1 to a learned model and sets a region output from the learned model to the candidate region R41. In addition, the setting function 34c inputs a bone density image collected from the object P at the measurement date/time T2 to the learned model and sets a region output from the learned model to the candidate region R41. Also, the setting function 34c inputs a bone density image collected from the object P at the measurement date/time T3 to the learned model and sets a region output from the learned model to the candidate region R41. That is, the setting function 34c sets the region output from the learned model to the candidate region R41 at each measurement date/time.

As an example, the setting function 34c executes morphology processing for a region output from a first learned model, thereby setting the candidate region R42 at each measurement date/time. Also, the setting function 34c sets a region output from a second learned model different from the first learned model to the candidate region R43 at each measurement date/time. As an example, the setting function 34c performs threshold-based processing, thereby setting the candidate region R44 at each measurement date/time. As an example, the setting function 34c performs graph cut processing, thereby setting the candidate region R45 at each measurement date/time.

For example, the output function 34e displays a three-axis graph formed from an axis representing the type (ROI TYPE) of the candidate region, an axis representing the value of the BMD, and an axis representing the measurement date/time, as shown in FIG. 8. The user selects a region of interest by referring to BMDs calculated in the past in addition to the BMDs calculated this time. In other words, the user can select a region of interest in consideration of the performance and reliability of each candidate region.

Here, the output function 34e may detect an abnormal value candidate from the plurality of analysis results. For example, in FIG. 8, the BMD corresponding to the candidate region R44 set at the measurement date/time T3 has a remarkably high value. In this case, the output function 34e detects the BMD for (R44, T3) as an abnormal value candidate. For example, the output function 34e calculates a standard deviation for the value of the BMD at each measurement date/time in each candidate region, and sets an error range based on the standard deviation. As an example, if a plurality of vertebral bodies (vertebrae) are included in a bone density image, the output function 34e performs statistical processing for the BMD calculated for each vertebral body, thereby setting the error range. As another example, the output function 34e can estimate the number of photons based on the pixel values of a bone density image and set the error range based on the estimation result. For example, the output function 34e estimates a quantum noise amount according to a Poisson distribution based on the estimation result of the number of photons and adds the quantum noise amount and a predetermined noise amount corresponding to circuit noise, thereby setting the error range. If the value of a BMD is not included in the error range, the output function 34e detects it as an abnormal value candidate. As an example, the output function 34e detects, as an abnormal value candidate, the value of a BMD that is not included in a predetermined section (for example, a range of 3σ) on a probability distribution.

Alternatively, the output function 34e may detect an abnormal value candidate for each measurement date/time. For example, the output function 34e calculates, for each measurement date/time, a standard deviation for the value of the BMD in each candidate region, and compares this with the error range, thereby detecting an abnormal value candidate. Alternatively, the output function 34e may detect an abnormal value candidate for each candidate region. For example, the output function 34e calculates, for each candidate region, a standard deviation for the value of the BMD at each measurement date/time, and compares this with the error range, thereby detecting an abnormal value candidate.

Note that the error range may be set based on a smaller candidate region. That is, the smaller the candidate region is, the lower the possibility that an inappropriate region is included in the candidate region is. For example, when setting a region of interest corresponding to a bone, if the candidate region is small, the possibility that a soft tissue or the like is included in the candidate region becomes low. On the other hand, the smaller the candidate region is, the smaller the number of pixels included in the candidate region is. Hence, it is readily affected by a statistical error. Here, the output function 34e can improve the detection accuracy of the abnormal value candidate by applying the error range set based on a smaller candidate region to other candidate regions.

A case where the candidate region R41 shown in FIG. 8 is smallest, and the size becomes large in the order of the candidate region R42, the candidate region R43, the candidate region R44, and the candidate region R45 will be described below as an example. For example, the output function 34e sets the error range of each candidate region based on the candidate region R41. If the error of the candidate region R41 is σ based on measurement in the past, the specifications of the apparatus, and the like in advance, the error of a region R4X corresponds to “σ×area of region R41/area of region R4X”. Then, the output function 34e compares the BMD of each candidate region with the error range set for candidate region, thereby detecting an abnormal value candidate.

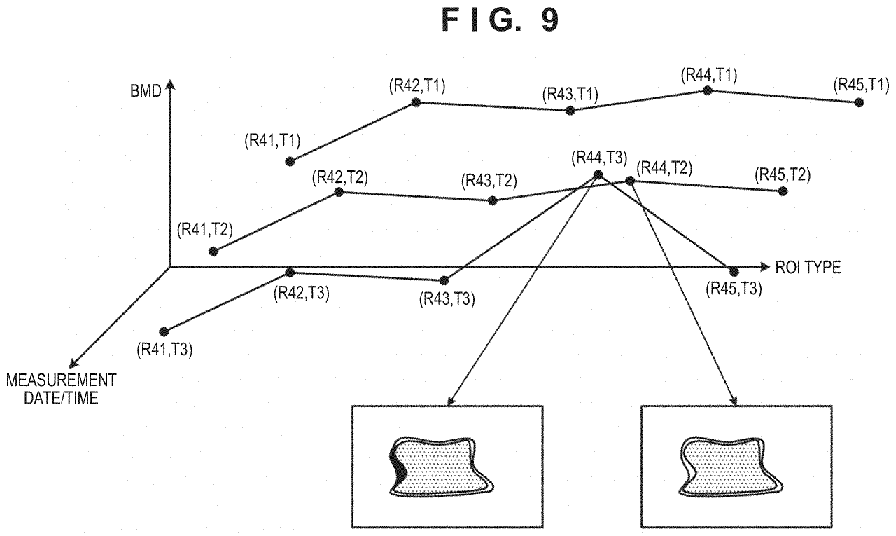

Also, if an abnormal value candidate is detected, the output function 34e may display an image representing the candidate region associated with the abnormal value candidate. For example, if the BMD for (R44, T3) is detected as an abnormal value candidate, the output function 34e displays an image representing the candidate region R44 set at the measurement date/time T3, as shown in FIG. 9. Furthermore, the output function 34e may display an image representing the candidate region R44 set at the measurement date/time T1 or T2, which is not detected as an abnormal value candidate, such that the images of the same candidate region at different measurement dates/times can be compared.

Furthermore, the output function 34e may color a portion where the bone density largely changes to highlight that portion. This allows the user to estimate the cause of the generation of the abnormal value candidate. For example, if there exist many portions where the bone density largely changes, it is estimated that the abnormal value candidate is generated because the bone density actually changes. On the other hand, if a portion where the bone density largely changes does not exist particularly, it is estimated that the abnormal value candidate is generated because the set candidate region is inappropriate.

Note that the display shown in FIG. 9 is merely an example, and various modifications can be made. For example, the output function 34e may provide an image display region in addition to the region to display the graph shown in FIG. 9, and display, in a stack, an image representing the candidate region R44 set at the measurement date/time T3 or an image representing the candidate region R44 set at the measurement date/time T2. Also, in FIG. 8 or 9, the description has been made assuming that a three-dimensional graph is displayed. However, the output function 34e may appropriately selectively display a two-dimensional graph. For example, based on a user operation, the output function 34e may selectively display a two-dimensional graph formed from two axes representing the type (ROI TYPE) of the candidate region and the value of the BMD and a two-dimensional graph formed from two axes representing the measurement date/time and the value of the BMD.

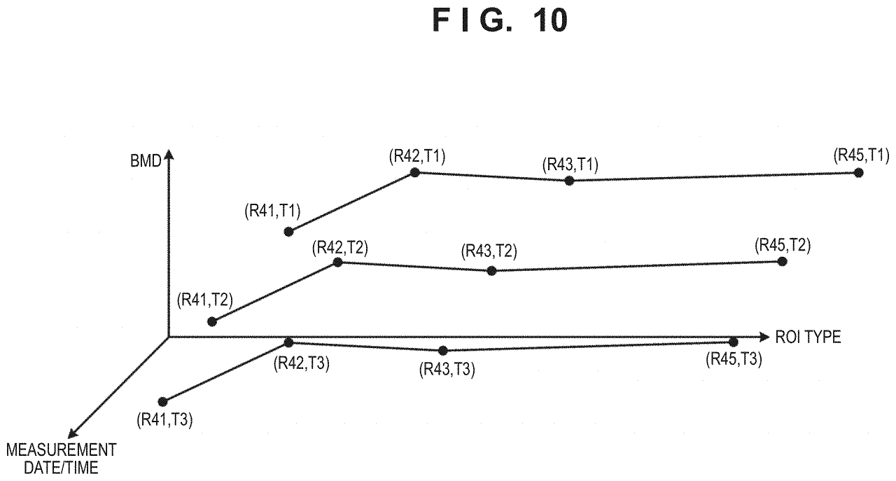

In addition, the output function 34e may changes the display target based on the abnormal value candidate. For example, if the BMD for (R44, T3) is detected as an abnormal value candidate, the output function 34e may not display the values of the BMDs corresponding to the candidate region R44, as shown in FIG. 10. Such change of the display target may be reflected from the measurement date/time T3 at which the abnormal value candidate is detected, or may be reflected from the next time on. If each candidate region is used as learning data of machine learning, the candidate region detected as the abnormal value candidate may be excluded from the learning data. Note that FIG. 10 is a view showing a display example according to the first embodiment.

Note that a case where a region of interest corresponding to a vertebra is set has been described above. This can similarly be applied to a case where a plurality of regions of interest are set. As an example, the setting function 34c sets a candidate region R211 and a candidate region R212 as the candidates of a region of interest corresponding to the vertebra. Also, the setting function 34c sets a candidate region R221 and a candidate region R222 as the candidates of a region of interest corresponding to a neural region. Also, the setting function 34c sets a candidate region R231 and a candidate region R232 as the candidates of a region of interest corresponding to a soft tissue. That is, to set a plurality of regions of interest, the setting function 34c sets a plurality of candidate regions in correspondence with each of the regions of interest.

Next, the analysis function 34d executes bone mineral density measurement for each combination of candidate regions. For example, the analysis function 34d performs BMD calculation based on the combination of the candidate region R211, the candidate region R221, and the candidate region R231. Also, the analysis function 34d performs BMD calculation based on the combination of the candidate region R211, the candidate region R221, and the candidate region R232. The analysis function 34d also performs BMD calculation based on the combination of the candidate region R211, the candidate region R222, and the candidate region R231. The analysis function 34d also performs BMD calculation based on the combination of the candidate region R211, the candidate region R222, and the candidate region R232. The analysis function 34d also performs BMD calculation based on the combination of the candidate region R212, the candidate region R221, and the candidate region R231. The analysis function 34d also performs BMD calculation based on the combination of the candidate region R212, the candidate region R221, and the candidate region R232. The analysis function 34d also performs BMD calculation based on the combination of the candidate region R212, the candidate region R222, and the candidate region R231. The analysis function 34d also performs BMD calculation based on the combination of the candidate region R212, the candidate region R222, and the candidate region R232.

Next, the output function 34e outputs the analysis results. For example, the output function 34e displays each BMD calculated based on the combination of the candidate regions in association with the combination of the candidate regions used to calculate the BMD. For example, the output function 34e displays, on the display 32, a graph with an abscissa representing the combination of candidate regions and an ordinate representing the BMD. Then, the accepting function 34f accepts, from the user, an operation of selecting one of the plurality of candidate regions as a region of interest. For example, if the user selects the value of the BMD calculated based on the combination of the candidate region R211, the candidate region R221, and the candidate region R231, the accepting function 34f can accept the operation from the user, determining that the candidate region R211 is selected as a region of interest corresponding to a vertebra, the candidate region R221 is selected as the candidate of a region of interest corresponding to a neutral region, and the candidate region R231 is selected as the candidate of a region of interest corresponding to a soft tissue.

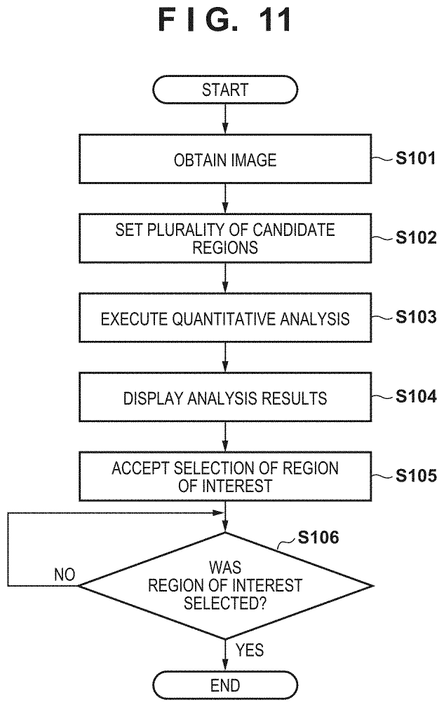

An example of the procedure of processing by the medical image processing apparatus 30 will be described next with reference to FIG. 11. FIG. 11 is a flowchart for explaining a series of procedures of processing of the medical image processing apparatus 30 according to the first embodiment. Step S101 corresponds to the obtaining function 34b. Step S102 corresponds to the setting function 34c. Step S103 corresponds to the analysis function 34d. Step S104 corresponds to the output function 34e. Step S105 and step S106 correspond to the accepting function 34f.

The processing circuit 34 obtain a medical image (step S101). For example, the processing circuit 34 generates a bone density image based on a first X-ray image and a second X-ray image. For example, the processing circuit 34 obtains a bone density image via the network NW. Next, the processing circuit 34 sets a plurality of candidate regions (step S102). Next, the processing circuit 34 executes quantitative analysis (step S103). For example, the processing circuit 34 performs bone mineral density (BMD) measurement for each of the plurality of candidate regions.

Next, the processing circuit 34 displays the analysis results (step S104). For example, the processing circuit 34 performs display shown in FIG. 6, 7, 8, 9, or 10, thereby displaying BMDs corresponding to the plurality of candidate regions on the display 32. Then, the processing circuit 34 accepts selection of a region of interest (step S105). That is, the processing circuit 34 accepts, from the user who has referred to the analysis result corresponding to each of the plurality of candidate regions in step S104, an operation of selecting one of the plurality of candidate regions as a region of interest.

Next, the processing circuit 34 determines whether the region of interest is selected (step S106). If the region of interest is not selected (NO in step S106), the processing circuit 34 shifts to a standby state. Alternatively, if the region of interest is not selected, the processing circuit 34 may return to step S102 again to set candidate regions again. Here, the processing circuit 34 may set candidate regions different from previous candidate regions by, for example, using a learned model different from the previous learned model or changing the threshold in threshold-based processing. On the other hand, if the region of interest is selected (YES in step S106), the processing circuit 34 ends the processing.

As described above, according to the first embodiment, the obtaining function 34b obtains a medical image. The setting function 34c sets a plurality of candidate regions on the medical image as the candidates of a region of interest to be set on the medical image. The analysis function 34d executes quantitative analysis concerning the composition of the object P for each of the plurality of candidate regions. The output function 34e outputs a plurality of analysis results corresponding to the plurality of candidate regions.

Hence, the medical image processing apparatus 30 according to the first embodiment can enable setting of an appropriate region of interest. More specifically, the user can refer to the plurality of analysis results corresponding to the plurality of candidate regions and determine which one of the plurality of candidate regions should be selected as a region of interest. That is, the user can select a more appropriate region of interest by using, as materials for determination, not only the medical image itself and information such as the shape of a candidate region set on the medical image but also an analysis result obtained when the candidate region is used.

Also, as described above, when setting a plurality of regions of interest on the medical image, the setting function 34c sets a plurality of candidate regions for each of the region of interests. In addition, the analysis function 34d executes quantitative analysis for each combination of candidate regions. Hence, even when executing quantitative analysis using a plurality of regions of interest, like, for example, bone mineral density measurement, the medical image processing apparatus 30 can enable setting of an appropriate region of interest.

Also, as described above, if quantitative analysis is performed a plurality of times for the object P, the output function 34e displays a plurality of analysis results in association with the time base. For example, the output function 34e displays, on the display 32, a three-axis graph formed from an axis representing the type of the candidate region, an axis representing the value of the BMD, and an axis representing the measurement date/time, as shown in FIG. 8. Hence, the medical image processing apparatus 30 can enable setting of a more appropriate region of interest. More specifically, when determining which one of the plurality of candidate regions should be selected as a region of interest, the user further uses analysis results obtained using the candidate regions in the past as the materials for determination, thereby selecting a more appropriate region of interest.

In addition, as described above, according to the first embodiment, the output function 34e detects an abnormal value candidate from the plurality of analysis results. Hence, the medical image processing apparatus 30 can enable setting of a more appropriate region of interest. For example, if an abnormal value candidate is detected, the output function 34e can display an image representing a candidate region associated with the abnormal value candidate. This allows the user to determine whether the abnormal value candidate is generated because the bone density or the like actually changes or because the candidate region is inappropriate. Upon determining that the abnormal value candidate is generated because the candidate region is inappropriate, the user can avoid selection of such a candidate region as a region of interest.

Note that when executing bone mineral density measurement and performing the bone mineral density measurement a plurality of times for the object P, the medical image processing apparatus 30 may basically perform the above-described processing only in the first bone mineral density measurement. That is, in the first bone mineral density measurement, the setting function 34c sets a plurality of candidate regions, the analysis function 34d executes bone mineral density measurement for each of the plurality of candidate regions, and the output function 34e outputs a plurality of analysis results corresponding to the plurality of candidate regions. This allows the user to select an appropriate region of interest. Here, in the second or subsequent bone mineral density measurement, the region of interest set in the first bone mineral density measurement can be used. Hence, the medical image processing apparatus 30 can integrate analysis conditions in bone mineral density measurement performed a plurality of times and easily evaluate a change over time.

However, there is also assumed a case where because of the change of the bone density of the object P, or the like, it is not appropriate that the region of interest set in the first bone mineral density measurement is used in the second or subsequent bone mineral density measurement. In this case, the medical image processing apparatus 30 may perform the above-described processing in the second or subsequent bone mineral density measurement as well. That is, in the second or subsequent bone mineral density measurement as well, the setting function 34c may set a plurality of candidate regions, the analysis function 34d may execute bone mineral density measurement for each of the plurality of candidate regions, and the output function 34e may output a plurality of analysis results corresponding to the plurality of candidate regions. The medical image processing apparatus 30 may do a setting for performing the above-described processing in the second or subsequent bone mineral density measurement as well, or may perform the above-described processing in accordance with a request from the user. For example, first, the region of interest set in the first quantitative analysis is used even in the second or subsequent bone mineral density measurement, and a report is created. Here, if the user who has referred to the report determines that the values of the BMDs written in the report are not appropriate, the medical image processing apparatus 30 can additionally execute the above-described processing.

### Second Embodiment

In the above-described embodiment, the description has been made assuming that a bone density image is generated, and candidate regions are set. A setting function 34c may perform preprocessing for a bone density image before candidate region setting and set candidate regions on the bone density image after the preprocessing. For example, as the preprocessing, the setting function 34c executes cortical bone removal processing for the bone density image. More specifically, the setting function 34c extracts a region corresponding to a cortical bone in the bone density image, and replaces the extracted region with a background component or the like. If such preprocessing is performed, a candidate region is set based on a cancellous bone other than the cortical bone. In general, the change of the bone density is small in the cortical bone and large in the cancellous bone. For this reason, when a BMD is calculated based on the cancellous bone, more useful information can be provided to the user.

Also, in the above-described embodiment, a case where a learned model is formed by a convolutional neural network has been described. However, the learned model may be formed by another machine learning method. As another machine learning method, a method can be used in which a plurality of candidates of a region of interest are extracted by feature amount extraction using a digital filter or pattern matching, and the final region of interest is classified and estimated by a method such as boosting or support vector machine.

Also, in the above-described embodiment, bone mineral density measurement has been described as an example of quantitative analysis. However, the embodiment is not limited to this, and application to various kinds of quantitative analysis concerning the composition of an object P is possible.

For example, the analysis function 34d may execute breast density measurement as quantitative analysis concerning the composition of the object P and calculate a Breast Density (BD). In this case, a medical information processing system 1 includes, for example, a mammography apparatus as a medical image diagnosis apparatus 10. For example, the medical image diagnosis apparatus 10 captures the breast of the object P and collects a mammography image such as an MLO (Mediolateral-Oblique) image, a CC (Cranio-Caudal) image, or a tomosynthesis image. Also, an obtaining function 34b obtains a mammography image from an image storage apparatus 20 or the medical image diagnosis apparatus 10.

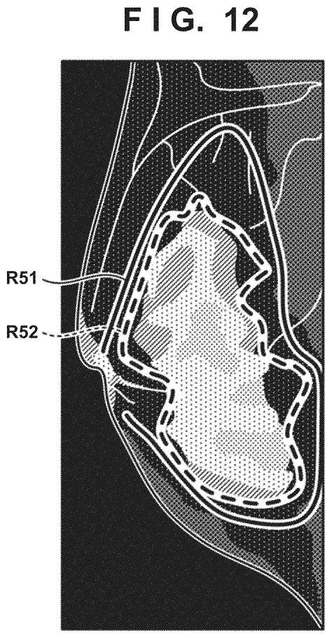

In breast density measurement, two regions of interest are set, like, for example, a region R51 of interest and a region R52 of interest in FIG. 12. The region R51 of interest is a region corresponding to a breast. The region R52 of interest is a region where a mammary gland exists. For example, the analysis function 34d can calculate a BD by dividing the area of the region R52 of interest by the area of the region R51 of interest. Here, as in the case of a BMD, if the region of interest is not appropriately set, the BD may have an inappropriate value. Note that FIG. 12 is a view for explaining breast density measurement according to the second embodiment.

For example, the setting function 34c sets a candidate region R511 and a candidate region R512 as the candidates of the region R51 of interest. Also, the setting function 34c sets a candidate region R521 and a candidate region R522 as the candidates of the region R52 of interest. That is, the setting function 34c sets a plurality of candidate regions for each of a plurality of regions of interest.

Next, the analysis function 34d performs BD calculation based on the combination of the candidate region R511 and the candidate region R521. Also, the analysis function 34d performs BD calculation based on the combination of the candidate region R511 and the candidate region R522. The analysis function 34d also performs BD calculation based on the combination of the candidate region R512 and the candidate region R521. The analysis function 34d also performs BD calculation based on the combination of the candidate region R512 and the candidate region R522. That is, the analysis function 34d executes breast density measurement for each combination of candidate regions.

Next, an output function 34e outputs the analysis results. For example, the output function 34e displays each BD calculated based on the combination of the candidate regions in association with the combination of the candidate regions used to calculate the BD. For example, the output function 34e displays, on a display 32, a graph with an abscissa representing the combination of candidate regions and an ordinate representing the BD.

Then, an accepting function 34f accepts, from the user, an operation of selecting one of the plurality of candidate regions as a region of interest. For example, if the user selects the value of the BD calculated based on the combination of the candidate region R511 and the candidate region R521, the accepting function 34f can accept the operation from the user, determining that the candidate region R511 is selected as the region R51 of interest, and the candidate region R521 is selected as the region R52 of interest.

Note that when executing breast density measurement and performing the breast density measurement a plurality of times for the object P, the medical image processing apparatus 30 may basically perform the above-described processing every time. That is, in the breast density measurement, the region of interest is basically set every time. For this reason, in every breast density measurement, the setting function 34c sets a plurality of candidate regions, the analysis function 34d executes breast density measurement for each of the plurality of candidate regions, and the output function 34e outputs a plurality of analysis results corresponding to the plurality of candidate regions.

Also, in the above-described embodiments, X-ray images such as a bone density image and a mammography image have been described as examples of a medical image. However, the embodiment is not limited to this. For example, application to quantitative analysis using a medical image such as an X-ray CT (Computed Tomography) image, a PET (Positron Emission computed Tomography) image, an SPECT (Single Photon Emission Computed Tomography) image, an ultrasonic image, or an MR (Magnetic Resonance) image is possible.

In FIG. 1, the places to install the medical image diagnosis apparatus 10, the image storage apparatus 20, and the medical image processing apparatus 30 are arbitrary if these can be connected via the network NW. For example, the medical image processing apparatus 30 may be installed in a hospital different from the medical image diagnosis apparatus 10. That is, the network NW may be formed by a local network closed in a hospital or may be a network via the Internet.

Also, in the above-described embodiments, the description has been made assuming that the medical image diagnosis apparatus 10 and the medical image processing apparatus 30 are separate apparatuses. However, the medical image processing apparatus 30 may be included in the medical image diagnosis apparatus 10. For example, a console device in the medical image diagnosis apparatus 10 may execute the function as the medical image diagnosis apparatus 10. In this case, the obtaining function 34b captures the object P, thereby obtaining a medical image.

The term “processor” used in the above explanation means, for example, a circuit such as a CPU, a GPU (Graphics Processing Unit), an Application Specific Integrated Circuit (ASIC), or a programmable logic device (for example, a Simple Programmable Logic Device (SPLD), a Complex Programmable Logic Device (CPLD), and a Field Programmable Gate Array (FPGA)). If the processor is, for example, a CPU, the processor implements the function by reading out a program stored in a storage circuit and executing it. On the other hand, if the processor is, for example, an ASIC, the function is directly incorporated as a logic circuit in the circuit of the processor, instead of storing the program in the storage circuit. Note that each processor according to the embodiments is not necessarily formed as a single circuit for each processor, and a plurality of independent circuits may be combined to form one processor to implement the function. Furthermore, a plurality of constituent elements in each drawing may be integrated into one processor to implement the function.

Also, in FIG. 1, the description has been made assuming that the single memory 33 stores a program corresponding to each processing function of the processing circuit 34. However, the embodiment is not limited to this. For example, a plurality of memories 33 may be distributedly arranged, and the processing circuit 34 may read out corresponding programs from the individual memories 33. Instead of storing the programs in the memory 33, the programs may directly be embedded in the circuit of the processor. In this case, the processor implements the functions by reading out the programs embedded in the circuit and executing these.

The constituent elements of the apparatuses according to the above-described embodiments are function-conceptual and need not always be physically formed as shown in the drawings. That is, a detailed form of dispersion/integration of the apparatuses is not limited to that shown in the drawings, and all or some of these can functionally or physically be dispersed/integrated in an arbitrary unit in accordance with various kinds of loads or a use situation. Also, all or some of the processing functions executed in each apparatus can be implemented by a CPU and a program analyzed and executed by the CPU, or implemented as hardware by a wired logic.

The medical image processing method described in the above embodiments can be implemented by a personal computer or a computer in a workstation executing a medical image processing program prepared in advance. The medical image processing program can be distributed via a network such as the Internet. Also, the medical image processing program can be recorded in a non-transitory computer-readable recording medium such as a hard disk, a flexible disk (FD), a CD-ROM, an MO, or a DVD and executed by a computer by reading out the program from the recording medium.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical image processing apparatus comprising:
a setting unit configured to set a plurality of candidate regions on a medical image as candidates of a region of interest to be set on the medical image;
an analysis unit configured to execute quantitative analysis concerning a composition of an object for each of the plurality of candidate regions; and
an output unit configured to output a plurality of analysis results and the plurality of candidate regions in association with each other.

2. The medical image processing apparatus according to claim 1, further comprising an accepting unit configured to accept, from a user who has referred to the plurality of analysis results, an operation of selecting one of the plurality of candidate regions as the region of interest.

3. The medical image processing apparatus according to claim 1, wherein
when setting a plurality of regions of interest on the medical image, the setting unit sets the plurality of candidate regions for each of the regions of interest, and
the analysis unit executes the quantitative analysis for each combination of the candidate regions.

4. The medical image processing apparatus according to claim 1, wherein the setting unit inputs the medical image to a learned model that is activated to accept input of the medical image and estimate the region of interest, and sets a region output from the learned model as an estimation result of the region of interest to one of the plurality of candidate regions.

5. The medical image processing apparatus according to claim 4, wherein the setting unit inputs the medical image to each of a plurality of learned models.

6. The medical image processing apparatus according to claim 1, wherein the setting unit performs threshold-based processing for a pixel value of the medical image, thereby setting at least one of the plurality of candidate regions.

7. The medical image processing apparatus according to claim 6, wherein the setting unit performs a plurality of threshold-based processes by changing a threshold, thereby setting at least two of the plurality of candidate regions.

8. The medical image processing apparatus according to claims 1, wherein the setting unit performs graph cut processing for the medical image, thereby setting at least one of the plurality of candidate regions.

9. The medical image processing apparatus according to claim 1, wherein the setting unit sets a region created by a user operation to one of the plurality of candidate regions.

10. The medical image processing apparatus according to claim 1, wherein the setting unit sets at least one of the plurality of candidate regions by morphology processing.

11. The medical image processing apparatus according to claim 1, wherein the output unit further displays images representing the candidate regions in association with the plurality of analysis results.

12. The medical image processing apparatus according to claim 1, wherein if the quantitative analysis is performed a plurality of times for the same object, the output unit displays the plurality of analysis results in association with a time base.

13. The medical image processing apparatus according to claim 1, wherein the output unit further detects an abnormal value candidate from the plurality of analysis results.

14. The medical image processing apparatus according to claim 13, wherein the output unit further displays an image representing the candidate region associated with the abnormal value candidate.

15. The medical image processing apparatus according to claim 14, wherein the output unit displays the image representing the candidate region such that a change over time before and after a point of time of occurrence of the abnormal value candidate can be identified.

16. The medical image processing apparatus according to claim 13, wherein the output unit sets an error range for each of the plurality of candidate regions based on a smaller candidate region and compares the analysis result corresponding to each of the plurality of candidate regions with the error range set for the candidate region, thereby detecting the abnormal value candidate.

17. The medical image processing apparatus according to claim 13, wherein the setting unit further adjusts a setting method of the candidate region based on the abnormal value candidate.

18. The medical image processing apparatus according to claim 1, wherein the analysis unit executes bone mineral density measurement or breast density measurement as the quantitative analysis.

19. A medical image processing method comprising:
setting a plurality of candidate regions on a medical image as candidates of a region of interest to be set on the medical image;
executing quantitative analysis concerning a composition of an object for each of the plurality of candidate regions; and
outputting a plurality of analysis results and the plurality of candidate regions in association with each other.

20. A non-transitory computer-readable storage medium storing a program for causing a computer to execute the method according to claim 19.
